(19) United States
(12) Patent Application Publication
(10) Pub. No.: US 2022/0151458 A1
(43) Pub. Date: May 19, 2022

(54) Footwear Sanitization Device

(71) Applicant: Bofill Holdings LLC, Opa Locka, FL (US)

(72) Inventor: Peter Bofill, Opa Locka, FL (US)

(21) Appl. No.: 16/953,266

(22) Filed: Nov. 19, 2020

(51) Int. Cl. A47L 23/02; A61L 2/10

(52) U.S. Cl. CPC A47L 23/02; A61L 2202/14; A61L 2/10

(57) ABSTRACT

A footwear sanitization device having a footwear chamber and a sanitization unit. The footwear chamber includes a first side wall, a second side wall, and a base arranged to form a channel. The footwear chamber may further include a cover and a posterior wall to form a more enclosed space. The sanitization unit includes a light assembly and an actuation mechanism. The light assembly is configured to emit sterilizing wavelengths of light, such as ultraviolet light, and is operably coupled to the actuation mechanism, such that when the actuation mechanism is triggered, the light assembly is switched from an off state to an on state. The light assembly is oriented to emit light within the channel of the footwear chamber. The sanitization unit may further include a timer for regulating the amount of light exposure and a processing unit for operably controlling the light assembly.

### CROSS-REFERENCES TO RELATED APPLICATIONS

None.

### STATEMENT REGARDING FEDERAL SPONSORED RESEARCH OR DEVELOPMENT

None.

### NAMES OF PARTIES TO A JOINT RESEARCH AGREEMENT None.

### REFERENCE TO A “SEQUENCE LISTING”, A TABLE, OR A COMPUTER PROGRAM LISTING APPENDIX SUBMITTED ON COMPACT DISC AND INCORPORATION-BY-REFERENCE OF THE MATERIAL ON THE COMPACT DISCLOSURE

None.

### STATEMENT REGARDING PRIOR DISCLOSURES BY AN INVENTOR OR JOINT INVENTOR

None.

### BACKGROUND OF THE INVENTION

### 1. Field of the Invention

The present invention relates to footwear, and more particularly, to an improved device and method for sanitizing footwear.

### 2. Description of Related Art Including Information Disclosed Under 37 CFR 1.97 And 37 CFR 1.98

Several designs for footwear sanitizers have been designed in the past. None of them, however, includes means for sanitizing footwear without the use of solvents or abrasives. While solvents offer a relatively cheap means for cleaning and sanitizing footwear, the solvents can actually end up damaging the footwear depending on the material of the footwear. If the instructions for using the solvents are not clearly visible to the user, the user may end up causing irreparable harm to the footwear. This may include staining the material of the footwear or it may shorten the lifespan of the material. The use of solvents is also untimely, as the solvent must be applied and then removed or allowed to dry. If abrasives such as a brush are used in conjunction with solvents or stand alone, the user must be careful in the amount of pressure that is applied. If too much pressure is applied or the bristles are too hard, then the material of the footwear may be scratched, scuffed, or otherwise disfigured.

Other patents describing the closest subject matter provide for a number of more or less complicated features that fail to solve the problem in an efficient and economical way. None of these patents suggest the novel features of the present invention.

A brief abstract of the technical disclosure in the specification and title are provided as well for the purposes of complying with 37 CFR 1.72 and are not intended to be used for interpreting or limiting the scope of the claims.

Without limiting the scope of the invention, a brief summary of some of the claimed embodiments of the invention is set forth below. Additional details of the summarized embodiments of the invention and/or additional embodiments of the invention may be found in the detailed description of the invention below.

### BRIEF SUMMARY OF THE INVENTION

It is one of the main objects of the present invention to provide a device for sanitizing the exterior of footwear.

It is another object of this invention to provide a footwear sanitization device which is free of solvents, abrasives, or other means that could damage the material of a pair of shoes.

It is still another object of the present invention to provide a means for sanitizing footwear using light irradiation and that is energy efficient.

It is yet another object of this invention to provide such a footwear sanitization device that is inexpensive to manufacture and maintain while retaining its effectiveness.

Further objects of the invention will be brought out in the following part of the specification, wherein detailed description is for the purpose of fully disclosing the invention without placing limitations thereon.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE SEVERAL VIEWS OF THE DRAWING

With the above and other related objects in view, the invention exists in the details of construction and combination of parts as will be more fully understood from the following description, when read in conjunction with the accompanying drawings in which:

FIG. 1 shows a front right perspective view of a footwear sanitization device, wherein a light assembly is configured to illuminate the interior of a footwear chamber.

FIG. 2 shows a top plan view of a footwear sanitization device, wherein the light assembly is integrated along the base of the footwear chamber.

FIG. 3 shows a front right perspective view of an alternative embodiment of a footwear sanitization device, wherein the footwear chamber includes a cover and posterior wall to completely encompass the footwear.

### DETAILED DESCRIPTION OF THE INVENTION

While this invention may be embodied in many different forms, there are described in detail herein specific embodiments of the invention. This description is exemplary of the principles of the invention and is not intended to limit the invention to the particular embodiments illustrated and described.

For the purpose of this disclosure, like reference numerals in the figures shall refer to like features unless otherwise indicated or is obvious by context.

The subject footwear sanitization device is sometimes referred to as the device, the invention, machine or other similar terms. These terms may be used interchangeably as context requires and from use the intent becomes apparent. The masculine can sometimes refer to the feminine and neuter and vice versa. The plural may include the singular and singular the plural as appropriate from a fair and reasonable interpretation in the situation.

Referring now to the drawings, where the present invention is generally referred to with numeral 10, it can be observed that it basically includes a footwear chamber 11 and a sanitization unit 20. The footwear chamber 11 is intended to receive one or more pieces of footwear at a time and generally comprises a base 12, a first side wall 13, and a second side wall 14.

Meanwhile, the sanitization unit 20 is intended to sterilize the footwear positioned within the footwear chamber 11. The sanitization unit 20 generally comprises, inter alia, a light assembly 21 and an actuation mechanism 22.

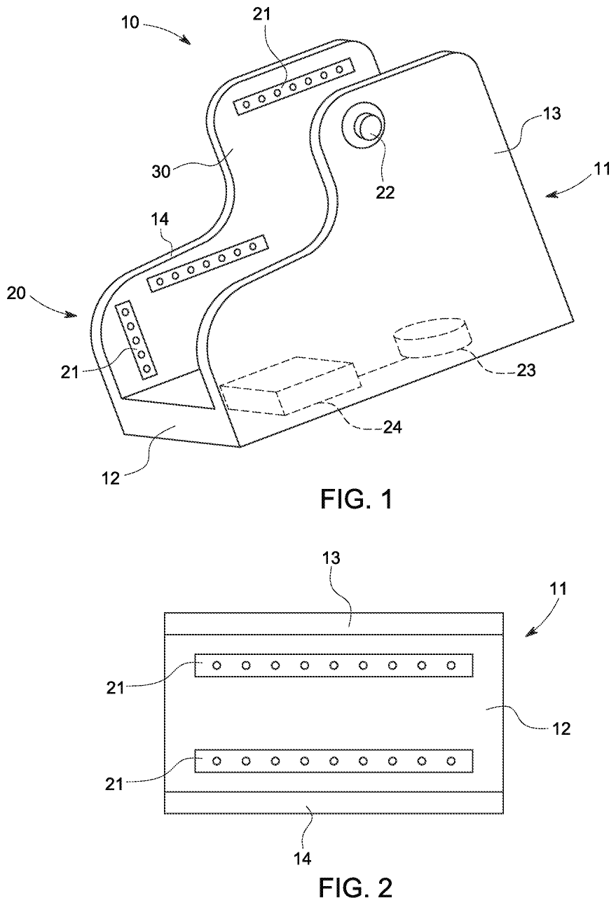

In reference to FIG. 1, the footwear chamber 11 is a walled structure, wherein the base 12, the first side wall 13, and the second side wall 14 form a channel for receiving one or more pieces of footwear. The first side wall 13 and the second side wall 14 are adjacently connected to the base 12, wherein the first side wall 13 and the second side wall 14 are positioned opposite each other across the base 12. The first side wall 13 and the second side wall 14 are spaced apart from each other to allow at least one piece of footwear between the first side wail 13 and the second side wall 14. The sanitization unit 20 is integrated with the footwear chamber 11, wherein that the light assembly 21 is configured to illuminate the channel in order to bathe the footwear in ultraviolet light.

The light assembly 21 may be integrated with one or more of the base 12, the first side wall 13, and the second side wall 14. The light assembly 21 is configured to emit sterilizing wavelengths of light, such as ultraviolet light. In the preferred embodiment, the light assembly 21 is configured to emit ultraviolet C light which is the best-known wavelength for effectively performing germicidal irradiation. However, different wavelengths may be used in other embodiments, as the different wavelengths may be found to be more effective depending on the material of the footwear or the environment in which the device is placed.

In general, the light assembly 21 comprises a plurality of lighting elements, wherein the plurality of lighting elements is distributed about the base 12, the first side wall 13, and the second side wall 14. Each of the plurality of lighting elements may be an incandescent bulb, fluorescent bulb, halogen bulb, light emitting diode, or any other suitable element capable of emitting irradiating and/or sterilizing light. The specific number of the plurality of lighting elements used depends on the type of light elements, the intensity of the light elements, and the placement of the light elements about the footwear chamber 11 so that the footwear may be completely sterilized on the exterior surface.

In one embodiment, the first side wall 13 and the second side wall 14 are spaced apart to accommodate a single piece of footwear between the first side wall 13 and the second side wail 14 at one time. The light assembly 21 is integrated along the base 12, the first side wall 13, and the second side wall 14 such that light is directed onto all exterior surfaces of the footwear. The first side wall 13 and the second side wall 14 are L-shaped to mimic the general profile of footwear. This allows for a more efficient placement of lighting elements to direct light onto the footwear. Further, this reduces the profile of the device and allows for a more compact device that is better suited for travel and storage in small spaces.

In another embodiment, the first side wall 13 and the second side wall 14 are spaced further apart in order to allow two pieces of footwear to be positioned between the first side wall 13 and the second side wall 14. The light assembly 21 is integrated along the base 12, the first side wall 13 and the second side wall 14 such that light is directed onto all exterior surfaces of the footwear. The first side wall 13 and the second side wall 14 are tall enough such that light may be directed onto the medial side of the opposing piece of footwear. While this would slightly increase the size in comparison to a device configured for a single piece of footwear, it would be more efficient in the expenditure of a user's time as they would be able to sanitize both pieces of footwear simultaneously while the user is standing in the footwear or whether the footwear is placed in the device while not being worn.

The light assembly 21 is integrated with the base 12 such that the soles of the footwear may be sanitized. The base 12 may have a transparent surface, wherein the light assembly 21 is mounted below the transparent surface such that the transparent surface protects the light assembly 21 from being damaged by the footwear. Preferably the transparent surface is fully transparent, thus allowing a maximum amount of light to pass through. In this way, when an individual steps onto the base 12, or otherwise places footwear on the base 12, the light assembly 21 is able to irradiate the soles of the footwear. It is also possible for the transparent surface to be semi-transparent, so long as sufficient light is able to pass through the transparent surface such that the soles of the footwear are effectively irradiated.

The light assembly 21 is integrated with the first side wall 13 and the second side wall 14 such that the sides and top of the footwear may be sanitized. Similar to the base 12, the first side wall 13 and the second side wail 14 may each have a transparent surface, wherein the light assembly 21 is mounted behind the transparent surface of the first side wall 13 and the second side wall 14. In this way, the transparent surface of the first side wall 13 and the second side wall 14 protects the light assembly 21 from being damaged by the footwear.

Preferably the transparent surface of the first side wall 13 and the transparent surface of the second side wall 14 are fully transparent, thus allowing a maximum amount of light to pass through. In this way, when an individual steps into the footwear chamber 11, or otherwise places footwear in the footwear chamber 11, the light assembly 21 is able to irradiate the sides and top of the footwear. It is also possible for the transparent surface of the first side wall 13 and the second side wall 14 to be semi-transparent, so long as sufficient light is able to pass through the transparent surface of the first side wall 13 and the second side wall 14 such that the sides and top of the footwear are effectively irradiated.

The term irradiation is intended to include a radiant light source in the sense that the light is emitted and shines. This may be a visible light and/or a wavelength not humanly visible. The light is preferably an effective wavelength or combination of wavelengths that provide at least a minimal sterilization effect. A more efficient sterilization is generally preferred.

In other embodiments, the light assembly 21 may be mounted to the medial side of the first side wall 13 and the medial side of the second side wall 14. The light assembly 21 is mounted onto to the first side wall 13 in a fixed position, such that light is directed onto the sides and top of the footwear. Similarly, the light assembly 21 is mounted onto to the second side wail 14 in a fixed position, such that light is directed onto the sides and top of the footwear.

The footwear chamber 11 may further comprise one or more reflective surfaces 30. The reflective surfaces 30 can be integrated with the top of the base 12, the medial side of the first side wall 13, the medial side of the second side wall 14, or otherwise positioned within the channel such that the reflective surfaces 30 reflect the light emitted by the light assembly 21. In this way, the number of lighting elements of the light assembly 21 may be reduced, which in effect reduces energy consumption and may reduce manufacturing costs.

The footwear chamber 11 may further comprise a mid-wall. The mid-wall is adjacently connected to the base 12 and positioned in between the first side wall 13 and the second side wall 14, such that the channel is partitioned into a first chamber and a second chamber. In one embodiment, the first chamber and the second chamber are each sized to receive one piece of footwear. The light assembly 21 is integrated along the base 12, the first side wall 13, the second side wall 14, and both sides of the mid-wall such that light is directed onto all exterior surfaces of the footwear. The first side wall 13, the second side wall 14, and the mid-wall are L-shaped to mimic the general profile of footwear. This reduces the profile of the device and allows for a more efficient placement of lighting elements to direct light onto the footwear. Further, this is more efficient in the user's time as both pieces of footwear can be sanitized simultaneously.

In another embodiment, the first chamber and the second chamber are each sized to receive two pieces of footwear. The light assembly 21 is integrated along the base 12, the first side wall 43, the second side wall 44, and both sides of the mid-wall such that light is directed onto all exterior surfaces of the footwear. The first side wall 13, the second side wall 14, and the mid-wall are tall enough such that light may be directed onto the medial side of the opposing piece of footwear. This type of design would allow multiple users to sanitize their footwear at the same time, which could be beneficial to have in public spaces.

The mid-wall may have a first transparent surface and a second transparent surface, wherein the light assembly 21 is mounted between the first transparent surface and the second transparent surface. In this way, the first transparent surface and the second transparent surface protect the light assembly 21 from being damaged by the footwear. Preferably the first transparent surface and the second transparent surface are fully transparent, thus allowing a maximum amount of light to pass through. In this way, when an individual steps into the footwear chamber 11, or otherwise places footwear in the footwear chamber 11, the light assembly 21 is able to irradiate the sides and top of the footwear. It is also possible for the first transparent surface and the second transparent surface to be semi-transparent, so long as sufficient light is able to pass through the first transparent surface and the second transparent surface such that the sides and top of the footwear are effectively irradiated.

In some embodiments, the one or more reflective surfaces 30 may also be integrated with the mid-wall. The reflective surfaces 30 may be positioned about both sides of the mid-wall. In this way, the reflective surfaces 30 reflect the light emitted by the light assembly 21 within both the first chamber and the second chamber.

The light assembly 21 is electrically connected to the actuation mechanism 22, wherein the actuation mechanism 22 toggles the light assembly 21 between an on state and an off state. In the on state, the light assembly 21 is emits ultraviolet C light, or another irradiating wavelength of light. The actuation mechanism 22 is integrated with the footwear chamber 11 such that the actuation mechanism 22 may be engaged by a user or such that the actuation mechanism 22 is able to detect the presence of the user or footwear. When the actuation mechanism 22 is engaged or detects the presence of the user or footwear, the light assembly 21 is toggled to the on state. Subsequently, when the actuation mechanism 22 is engaged a second time or the presence of the user or footwear is no longer detected, the light assembly 21 is toggled to the off state.

In some embodiments, the actuation mechanism 22 is a push button, flip switch, or similar mechanism, wherein the actuation mechanism 22 is engaged a first time to switch the light assembly 21 to the on state and engaged a second time to switch the light assembly 21 to the off state. Preferably the actuation mechanism 22 is integrated with the exterior of the footwear chamber 11 such that a user may engage the actuation mechanism 22 with their foot. In this way, the user does not need to bend over or dirty their hand. However, it is also possible for the actuation mechanism 22 to be integrated with the exterior of the footwear chamber 11 such that a user may engage the actuation mechanism 22 with their hand.

In other embodiments, the actuation mechanism 22 may be a pressure switch, or similar device, wherein the actuation mechanism 22 must be constantly engaged to maintain the light assembly 21 in the on state. Preferably the actuation mechanism 22 is integrated within the channel of the footwear chamber 11 in such embodiments, wherein the actuation mechanism 22 is engaged by the footwear when the footwear is positioned within the channel. For example, on the bottom surface of the footwear chamber 11 so that the weight of the footwear or that of the user standing on the switch activates the light assemblies 21. When the footwear is removed from the channel, the actuation mechanism 22 is subsequently disengaged and the light assembly 21 is powered off.

In some embodiments, the sanitization unit 20 may further comprise a timer 23 to automatically switch the light assembly 21 from the on state to the off state. In this way, the user must only engage the actuation mechanism 22 a single time. Further, this limits the duration for which the light assembly 21 is switched to the on state and thus increases the energy efficiency of the present invention. The timer 23 is electrically connected with the light assembly 21 such that the timer 23 breaks the circuit after a preset time in order to toggle the light assembly 21 from the on state to the off state, for example after several seconds or minutes, depending on the power of the lights. In some embodiments the timer 23 may be integrated with the actuation mechanism 22, wherein the timer 23 flips the actuation mechanism 22 in order to break the circuit. In other embodiments, the timer 23 may have its own independent means for breaking the circuit.

The sanitization unit 20 may further comprise a processing unit 24, wherein the processing unit 24 is electronically connected to the actuation mechanism 22 and the light assembly 21. The actuation mechanism 22 and the processing unit 24 are configured to detect the presence of a user or footwear. When the presence of the user or footwear is detected, the processing unit 24 toggles the light assembly 21 to the on state.

In one embodiment the actuation mechanism 22 is a motion sensor. The processing unit 24 may be configured to only toggle the light assembly 21 to the on state when motion is detected within a close range to the actuation mechanism 22, so as to avoid unnecessarily enabling the light assembly 21, for example, when someone is simply walking past the device. Preferably the actuation mechanism 22 is positioned within the channel of the footwear chamber 11 in such embodiments; however, it is also possible for the actuation mechanism 22 to be positioned about the exterior of the footwear chamber 11.

In another embodiment the actuation mechanism 22 is a camera. The actuation mechanism 22 records the area surrounding the footwear chamber 11, while the processing unit 24 analyzes the captured frames to determine if a user or footwear is approaching the channel of the footwear chamber 11. The processing unit 24 may use software that identifies preset motions such as a leg extending. When the processing unit 24 detects a match with one of the preset motions, the processing unit 24 toggles the light assembly 21 to the on state.

It is also possible for the processing unit 24 to utilize artificial intelligence to enhance the detection of a user or footwear towards the footwear chamber 11, thus reducing incidents of prematurely enabling the light assembly 21 and reducing energy costs. Furthermore, the artificial intelligence could be used to identify movements associated with specific individuals and reduce the incidents of prematurely enabling the light assembly 21 even more.

In yet another embodiment, the actuation mechanism 22 is a transceiver. The actuation mechanism 22 allows the processing unit 24 to communicate with an external device, such as a smartphone or near field communication tag. When the processing unit 24 detects the presence of the external device via the actuation mechanism 22, the processing unit 24 switches the light assembly 21 to the on state.

When the actuation mechanism 22 is a transceiver, the external device can also be used to remotely adjust settings of the sanitization unit 20. A control application may be installed on the external device, wherein a user is able to manipulate or monitor settings for the light assembly 21 remotely through the control application For example, the control application may allow the user to adjust the intensity of the light assembly 21, toggle the light assembly 21 between the on state and the off state, or monitor the electricity consumption of the light assembly 21.

In some embodiments, the light assembly 21 may comprise one or more operable mounts, wherein each of the operable mounts supports one or more lighting elements. The operable mounts may allow for simple movement of the lighting elements, such as a pivoting motion, or more advanced motion, such as moving along a track, wherein the lighting elements can be relocated within the footwear chamber 11. Each of the operable mounts is electronically connected to the processing unit 24 such that the processing unit 24 may adjust the direction of the light emitted from the lighting element. In this way, the light assembly 21 can be targeted to specific regions of the footwear chamber 11. This also allows the light assembly 21 to be used in a scanning manner, wherein the number of lighting elements can be reduced. For example, instead of a lighting element being mounted on the base 12, the first side wall 13, and the second side wall 14, a single lighting element can be mounted to a track that runs across the footwear chamber 11; the single lighting element can then be moved side to side in order to irradiate the top, sides, and bottom of the footwear.

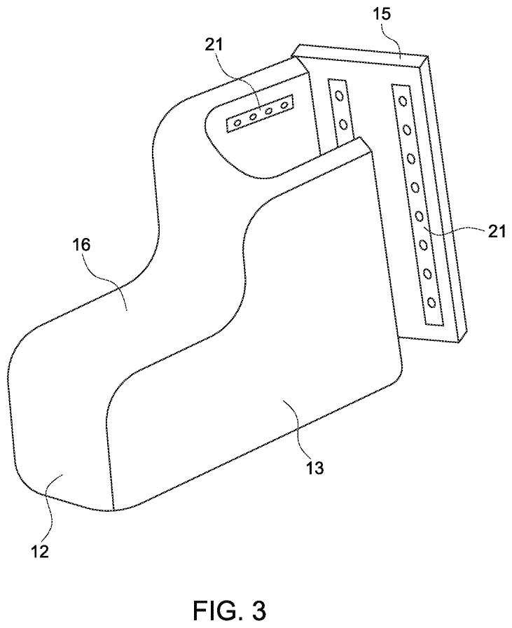

In reference to FIG. 3, in some embodiments the footwear chamber 11 further comprises a cover 16. The cover 16 is adjacently connected to the first side wall 13 and the second side wall 14 opposite to the base 12, such that the cover 16 forms the top of the footwear chamber 11. The cover 16 may also extend down to cover 16 the anterior of the footwear chamber 11. The cover 16 forms a more enclosed space, thus preventing light from leaking out of the channel of the footwear chamber 11. This prevents the user from being exposed to the irradiating light and can also increase efficiency of the light assembly 21.

The light assembly 21 may also be integrated into the cover 16, such that light is directed onto the top of the footwear. The cover 16 may have a transparent surface, wherein the tight assembly 21 is mounted above the transparent surface. In this way, the transparent surface protects the light assembly 21 from being damaged by the footwear. Preferably the transparent surface is fully transparent, thus allowing a maximum amount of light to pass through. In this way, when an individual steps into the footwear chamber 11, or otherwise places footwear in the footwear chamber 11, the light assembly 21 is able to irradiate the top of the footwear. It is also possible for the transparent surface to be semi-transparent, so long as sufficient light is able to pass through the transparent surface such that the top of the footwear is effectively irradiated.

In some embodiments, the one or more reflective surfaces 30 may also be integrated with the cover 16. In this way, lighting elements on the base 12, the first side wall 13, or the second side wall 14 can be angled upward at the cover 16, wherein the one or more reflective surfaces 30 directs light onto the top of the footwear. This allows the number of lighting elements to be reduced.

In further reference to FIG. 3, the footwear chamber 11 may further comprise a posterior wall 15. The posterior wall 15 is connected adjacent to the first side wall 13 or the second side wall 14, wherein the posterior wall 15 encloses the back of the footwear chamber 11. The posterior wall 15 may be hinged to the first side wall 13 or the second side wall 14, or otherwise removably attached to the first side wall 13 or the second side wall 14. This allows the posterior wall 15 to function as a door that may be opened and closed.

When the posterior wall 15 is configured as a door, the door may be manually opened by the user or automatically opened. If the door is configured to be automatic, the posterior wall 15 is connected to the first side wall 13 or the second side wall 14 via a door actuator, wherein the door actuator is able to pivot or otherwise move the posterior wall 15 between an open and closed position. The door actuator is electronically connected to the processor such that the processing unit 24 controls the door actuator to open and close the posterior wall 15 when a user or footwear is detected.

The light assembly 21 may also be integrated into the posterior wall 15, such that light is directed onto the backside of the footwear. The posterior wall 15 may have a transparent surface, wherein the light assembly 21 is mounted behind the transparent surface. In this way, the transparent surface protects the light assembly 21 from being damaged by the footwear. Preferably the transparent surface is fully transparent, thus allowing a maximum amount of light to pass through. In this way, when an individual steps into the footwear chamber 11, or otherwise places footwear in the footwear chamber 11, the light assembly 21 is able to irradiate the backside of the footwear. It is also possible for the transparent surface to be semi-transparent, so long as sufficient light is able to pass through the transparent surface such that the backside of the footwear is effectively irradiated.

In some embodiments, the one or more reflective surfaces 30 may also be integrated with the posterior wall 15. In this way, lighting elements on the base 12, the first side wall 13, the second side wall 14, or cover 16 can be angled backwards at the posterior wall 15, wherein the one or more reflective surfaces 30 directs light onto the backside of the footwear. This allows the number of lighting elements to be reduced.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

I claim:

1. A footwear sanitization device, the footwear sanitization device comprising:
a footwear chamber;
the footwear chamber comprising a base, a first side wall, and a second side wall;
the first side wall and the second side wall being adjacently connected to the base;
the first side wall being positioned opposite the second side wall across the base, wherein the first side wall, the second side wall, and the base form a channel;
a sanitization unit being integrated with the footwear chamber;
the sanitization unit comprising a light assembly and an actuation mechanism;
the light assembly being electrically connected to the actuation mechanism, wherein the light assembly can be toggled between an on state and an off state;
the light assembly being configured to illuminate the channel of the footwear chamber; and the light assembly being configured to emit ultraviolet light in the on state.

2. The footwear sanitization device as claimed in claim 1 wherein:
the sanitization unit further comprises a timer; the timer being electrically connected with the light assembly; and the timer being configured to toggle the light assembly from the on state to the off state after a preset time.

3. The footwear sanitization device as claimed in claim 1 wherein:
the sanitization unit further comprises a processing unit;
the processing unit being electronically connected to the actuation mechanism and the light assembly; and the processing unit toggling the light assembly to the on state when the actuation mechanism is triggered.

4. The footwear sanitization device claimed in claim 3, wherein the actuation mechanism is a motion sensor.

5. The footwear sanitization device as claimed in claim 3, wherein the actuation mechanism is a transceiver.

6. The footwear sanitization device as claimed in claim 3, wherein the actuation mechanism is a camera.

7. The footwear sanitization device as claimed in claim 1 wherein:
the footwear chamber further comprises a cover; the cover being connected adjacent to the first side wall and the second side wall opposite the base.

8. The footwear sanitization device as claimed in claim 1 wherein:
the footwear chamber further comprises a posterior wall;
the posterior wall being configurable between an open position and a closed position.
